(19) United States
(12) Patent Application Publication
(10) Pub. No.: US 2010/0209373 A1
(43) Pub. Date: Aug. 19, 2010

(54) DITHIOLS AND USE THEREOF FOR STRENGTHENING THE HAIR

(75) Inventors: Michel Philippe, Wissous (FR); Hervé Andrean, Paris (FR); Philippe Barbarat, Bois-Colombes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A., Paris (FR)

(21) Appl. No.: 12/705,073

(22) Filed: Feb. 12, 2010

Related U.S. Application Data
(63) Continuation of application No. 10/846,686, filed on May 17, 2004, now abandoned.
(60) Provisional application No. 60/477,728, filed on Jun. 12, 2003.

(30) Foreign Application Priority Data
May 16, 2003 (FR) 03 05899

(51) Int. Cl. A61K 8/46

(52) U.S. Cl. 424/70.1

(57) ABSTRACT

The disclosure relates to the use of dithiols for strengthening keratin materials, and to novel dithiols and to cosmetic compositions comprising them.

This application claims benefit of U.S. Provisional Application No. 60/477,728, filed Jun. 12, 2003, hereby incorporated by reference.

The present disclosure relates to the cosmetic use of dithiols for strengthening keratin materials. Also disclosed herein are novel dithiols and cosmetic compositions comprising them.

The term “keratin material” comprises the skin and keratin fibers. The term “keratin fibers” comprises hair on the head, eyelashes, eyebrows and other hairs. In one aspect, the present disclosure is directed to keratin fibers, for example, hair on the head.

Compounds containing a thiol functional group are known in the art and may be used in cosmetics as reducing agents for long-lasting shaping of the hair, especially in permanent-waving. These compounds, for example, thioglycolic acid and those disclosed in, for example, U.S. Pat. Nos. 2,719,813 and 2,719,814 and in published patent application WO 01/74318, tend to have drawbacks associated, for example, with degradation of the fibers.

Further, the use of certain dithiols is also known in the art. For example, French patent application No. 0,204,352, filed by the Applicant discloses a metallic coating process to give keratin fibers shampoo-fast cosmetic properties. According to the process, a composition containing at least one metallic salt is applied to keratin fibers, followed by application of a composition containing at least one reducing agent. Among the reducing agents disclosed are BMS (bis(mercaptoethyl) sulphone), DTT (dithiothreitol) and DMH (N,N′-dimethyl-N,N′-bis(mercaptoethyl)hydrazine).

French patent application No. 0,204,352 discloses generally the use of dithiols in a process comprising the application of a metallic salt and of a reducing agent to fix and/or hold the hair, for haircare, in shampoos, for hair conditioning, for example to give the hair softness, or alternatively for making up the hair. However, it does not disclose the specific use of dithiols for strengthening or increasing the rigidity of keratin fibers.

As used herein, the term “strengthening keratin materials” means improving the mechanical properties of keratin materials, which may be manifested, for example, as follows:
by a rigidification of the keratin materials, which may afford greater consistency and body and a more pleasant feel. This may result in an increase in the volume of the keratin fibers, greater styling ease and greater hold of the style, or
by greater resistance to mechanical tensile forces applied to the keratin materials, for example during combing. This greater resistance may be demonstrated, for example, on the hair of persons of African descent, on embrittled hair, and on sensitized hair. This increase in resistance may be referred to as a breaking-resistance effect.

Thus, it would be desirable to provide dithiols that can strengthen keratin materials, and be remanent on the keratin materials even after shampooing five times.

Accordingly, disclosed herein is the use of dithiols of formula (I), below, to strengthen keratin materials, for example keratin fibers:

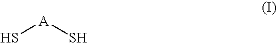

wherein A is chosen from
linear and branched, saturated and unsaturated C₁ to C₃₀ hydrocarbon-based chains optionally interrupted with at least one entity chosen from heteroatoms and functional groups such as

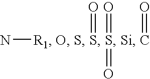

wherein R₁ is chosen from H, (C₁ to C₈)alkyl, (C₁ to C₈)acyl, (C₁ to C₈)alkyloxycarbonyl, halo, and (C₁ to C₈)alkylaminocarbonyl groups;
and from 5-, 6- and 7-membered aromatic and non-aromatic rings, and
from 5-, 6- and 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups.

According to one embodiment of the present disclosure, the compounds of formula (I) are used to increase the rigidification of keratin materials, for example the hair.

According to a second embodiment of the present disclosure, the compounds of formula (I) are used to give the hair a breaking-resistance effect, for example, on the hair of persons of African descent, on embrittled hair, and on sensitized hair.

Another embodiment of the present disclosure relates to the use of the dithiols of formula (II):

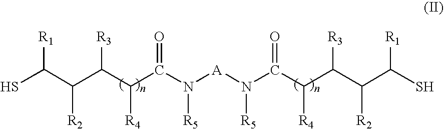

wherein each n is 0 or 1;
A is chosen from linear and non-linear, saturated and unsaturated C₁ to C₁₈ alkylene groups, optionally interrupted with at least one entity chosen from 5-, 6- or 7-membered aromatic and non-aromatic rings, and from heteroatoms or functional groups such as

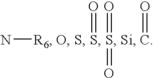

A may also be chosen from 5-, 6- and 7-membered aromatic and non-aromatic rings.
A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups;
R₁, R₂, R₃, and R₄ are each independently chosen from H, COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups; and
R₅ and R₆ are each independently chosen from H, (C₁ to C₄)alkyl, (C₁ to C₄)acyl, (C₁ to C₄)alkyloxycarbonyl, and (C₁ to C₄)alkylaminocarbonyl groups.

Exemplary compounds of formula (II) are shown below:

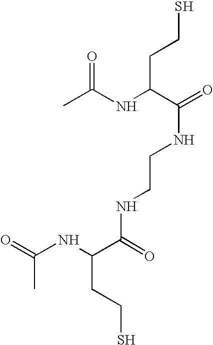

(1) 2-acetylamino-N-[2-(2-acetylamino-4-mercaptobutyrylamino)ethyl]-4-mercaptobutyramide

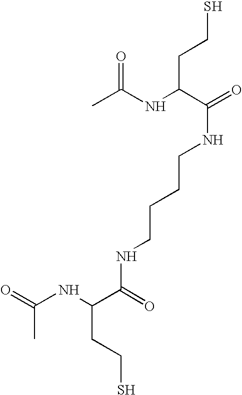

(2) 2-acetylamino-N-[2-(2-acetylamino-4-mercaptobutyrylamino)butyl]-4-mercaptobutyramide

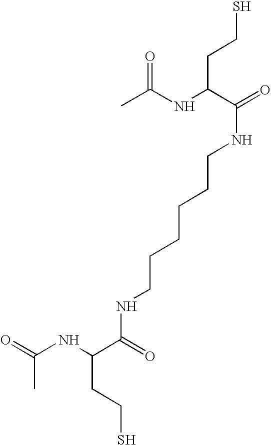

(3) 2-acetylamino-N-[2-(2-acetylamino-4-mercaptobutyrylamino)hexyl]-4-mercaptobutyramide

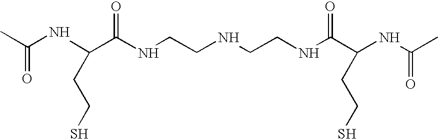

(4) 2-acetylamino-N-{2-[2-(2-acetylamino-4-mercaptobutyrylamino)ethylamino]ethyl}-4-mercaptobutyramide

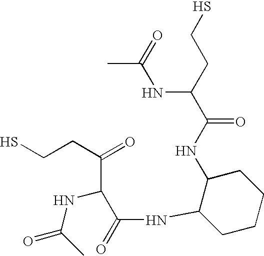

(5) 2-acetylamino-N-[2-(2-acetylamino-4-mercaptobutyrylamino)cyclohexyl]-4-mercaptobutyramide

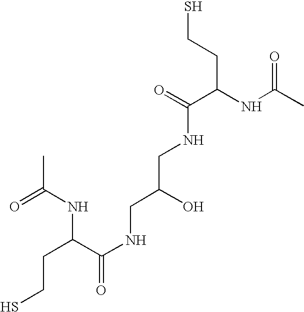

(6) 2-acetylamino-N-[2-(2-acetylamino-4-mercaptobutyrylamino)-2-hydroxypropyl]-4-mercaptobutyramide, and

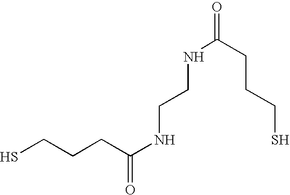

(7) 4-mercapto-N-[2-(4-mercaptobutyrylamino)ethyl]butyramide.

For example, the compound of formula (II) may be 2-acetylamino-N-{2-[2-(2-acetylamino-4-mercaptobutyrylamino)ethylamino]ethyl}-4-mercaptobutyramide.

The synthesis of the compounds of formula (II) may be performed as follows: a solution of a thiolactone is treated, under an inert atmosphere, with a diamine solution, the reaction temperature ranging from about 0 degrees Celsius to the reflux temperature of the solvent, for a duration ranging from about 15 minutes to about 12 hours.

Another embodiment of the present disclosure relates to the use of the dithiols of formula (III):

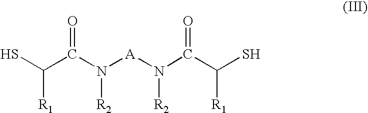

wherein A is chosen from linear and non-linear, saturated and unsaturated C₁ to C₁₈ alkylene groups, optionally interrupted with at least one entity chosen from 5-, 6- or 7-membered aromatic and non-aromatic rings and from heteroatoms or functional groups such as:

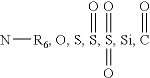

A may also be chosen from 5-, 6- or 7-membered aromatic and non-aromatic rings.
A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups; and
R₁ and R₂ are each independently chosen from H and C₁ to C₈ alkyl, and
R₆ is chosen from H, (C₁ to C₄)alkyl, (C₁ to C₄)acyl, (C₁ to C₄)alkyloxycarbonyl, and (C₁ to C₄)alkylaminocarbonyl groups.

One embodiment of the present disclosure relates to cosmetic compositions comprising at least one dithiol of formula (II) in a cosmetically acceptable medium.

The compositions of the disclosure may be for cosmetic or pharmaceutical use. For example, the compositions may be for cosmetic use. Thus, the compositions may comprise a non-toxic physiologically acceptable medium that can be applied to, for example, human skin, integuments, and lips. As used herein, the term “cosmetic” means a composition of pleasant appearance, odor and feel.

Compositions for application to the scalp or the hair may be in the form of haircare lotions, for example, for daily or twice-weekly application, shampoos or hair conditioners, for example for twice-weekly or weekly application, liquid or solid soaps for cleansing the scalp, for example for daily application, hair-shaping products (for example lacquers, hair setting products and styling gels), treating masks, and creams or foaming gels for cleansing the hair. They may also be in the form of hair dyes or hair mascaras to be applied, for example, with a brush or a comb.

In one embodiment, the compositions disclosed herein may comprise at least one dithiol selected from compounds (1), (2), (3), (4), (5), (6), and (7).

The compositions according to the present disclosure may also comprise cosmetic additives and/or formulation adjuvants including, but not limited to anionic, cationic, amphoteric or nonionic surfactants; nacreous agents; opacifiers; pigments and dyes; oils; waxes, including ceramides; organic or mineral UV-screening agents; free-radical scavengers; vitamins; proteins; antidandruff agents; plasticizers; agents for modifying and setting the pH; antioxidants; preserving agents; hair dye precursors; and oxidizing agents.

The compositions may be packaged in aerosol devices in the presence of one or more propellants. These propellants may be chosen from, for example, dimethyl ether, C₃₋₅ alkanes, 1,1-difluoroethane, mixtures of dimethyl ether and of C₃₋₅ alkanes and mixtures of 1,1-difluoroethane and of dimethyl ether and/or of C₃₋₅ alkanes.

A person skilled in the art will be able to select the optional additives and the amount thereof such that they do not harm the advantageous properties of the compositions of the present disclosure using routine skill.

The compositions may be in the form of, for example, thickened fluids, gels, creams, mousses, water-in-oil emulsions, oil-in-water emulsions or multiple emulsions.

The compositions may be applied to, for example, the hair, the skin, the eyelashes, the eyebrows and the nails.

The invention is illustrated in greater detail by the example described below. Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE

Seven compounds corresponding to formula (II) were prepared. These compounds were characterized by proton NMR, mass spectrometry, and a thiol function assay. These compounds were each prepared from an amine and homocysteine N-acetylthiolactone or from an amine and butyrothiolactone, following a similar procedure.

### Synthesis of 2-acetylamino-N-[2-(2-acetylamino-4-mercaptobutyrylamino)ethyl]-4-mercaptobutyramide (1)

A solution of ethylenediamine in dichloromethane (10⁻² mol in 15 ml) was added over ten minutes under an argon atmosphere, to a solution of homocysteine N-acetylthiolactone in dichloromethane (2×10⁻² mol in 35 ml), with magnetic stirring and at room temperature. After two hours, the reaction medium set to a solid, and was maintained at the reflux temperature of the solvent for two hours and cooled. The precipitate was filtered off and washed with dichloromethane, suction-drained and dried on a rotary evaporator, to yield 3.5 grams of odor-free white powder.

Theoretical mass 3.79; 93% yield.

Compounds (2) to (7) were prepared using the amines shown in Table 1 following analogous techniques.

TABLE 1

| | | Homocysteine | | |
|---|---|---|---|---|
| | | N-acetylthio- | | Yield |
| Dithiol | Amine | lactone | | Y (%) |
| (2) | 1,4-diaminobutane | 6 × 10⁻² | mol | 91.3 |
| | 3 × 10⁻² mol | | | |
| (3) | 1,6-diaminohexane | 6 × 10⁻² | mol | 92.5 |
| | 3 × 10⁻² mol | | | |
| (4) | diethylenetriamine | 0 × 36 | mol | 99 |
| | 0 × 18 mol | | | |
| (5) | trans-1,2-diaminocyclohexane | 0 × 2 | mol | 74 |
| | 0 × 1 mol | | | |
| (6) | 1,3-diaminopropanol | 0 × 1 | mol | 93.5 |
| | 5 × 10⁻² mol | | | |
| (7) | ethylenediamine | 0 × 1 | mol | 81 |
| | 5 × 10⁻² mol | | | |

Each of the dithiols (1) to (7) above was introduced into a cosmetic composition comprising from 1% to 35%, for example from 5% to 15% of dithiol, and from 70% to 95% of water. The compositions were applied to various hair types, and a known fixing step was optionally performed after applying the composition. A panel of experts judged the treated hair as being strengthened relative to untreated hair, and that the strengthening of which was still detectable after shampooing five times.

What is claimed is:

1. A method of strengthening a keratin material comprising applying to the keratin material at least one dithiol of formula (I): 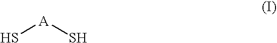
wherein A is chosen from
linear and branched, saturated and unsaturated C₁ to C₃₀ hydrocarbon-based chains which are optionally interrupted with at least one entity chosen from heteroatoms, functional groups, and 5-, 6- and 7-membered aromatic and non-aromatic rings, and
5-, 6- and 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups.

2. The method of claim 1 wherein the keratin material is a keratin fiber.

3. The method of claim 1, wherein the C₁ to C₃₀ hydrocarbon-based chains of A are interrupted with at least one functional group chosen from: 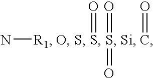
wherein R₁ is chosen from H, (C₁ to C₈)alkyl, (C₁ to C₈)acyl, (C₁ to C₈)alkyloxycarbonyl, (C₁ to C₈)alkylaminocarbonyl, and halo groups.

4. A method of strengthening a keratin material comprising applying to the keratin material at least one dithiol of formula (II): 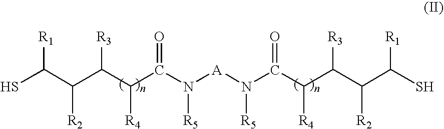
wherein each n, which may be identical or different, is chosen from 0 and 1;
A is chosen from
linear and non-linear, saturated and unsaturated C₁ to C₁₈ alkylene groups, optionally interrupted with at least one entity chosen from 5-, 6- and 7-membered aromatic and non-aromatic rings, heteroatoms and functional groups; and
5-, 6- and 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups;
R₁, R₂, R₃, and R₄, which may be identical or different, are chosen from H, COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups; and
R₅, which may be identical or different, is chosen from H, (C₁ to C₄)alkyl, (C₁ to C₄)acyl, (C₁ to C₄)alkyloxycarbonyl, and (C₁ to C₄)alkylaminocarbonyl groups.

5. The method of claim 4, wherein the keratin material is a keratin fiber.

6. The method of claim 4, wherein the C₁ to C₁₈ alkylene groups of A are interrupted by at least one heteroatom or functional group chosen from: 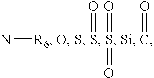
wherein R₆ is chosen from H, (C₁ to C₄)alkyl, (C₁ to C₄)acyl, (C₁ to C₄)alkyloxycarbonyl, and (C₁ to C₄)alkylaminocarbonyl groups.

7. A method of strengthening a keratin material comprising applying to the keratin material at least one dithiol chosen from: 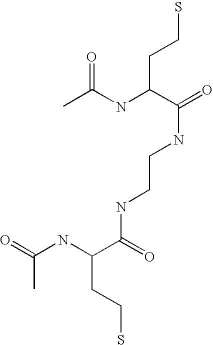
(1) 2-acetylamino-N-[2-(2-acetylamino-4-mercaptobutyrylamino)ethyl]-4-mercaptobutyramide, 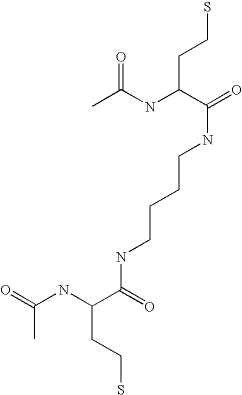
(2) 2-acetylamino-N-[2-(2-acetylamino-4-mercaptobutyrylamino)butyl]-4-mercaptobutyramide, 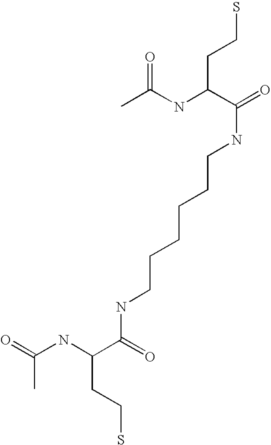
(3) 2-acetylamino-N-[2-(2-acetylamino-4-mercaptobutyrylamino)hexyl]-4-mercaptobutyramide, 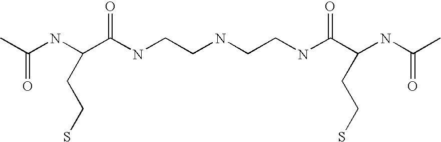
(4) 2-acetylamino-N-{2-[2-(2-acetylamino-4-mercaptobutyrylamino)ethylamino]ethyl}-4-mercaptobutyramide, 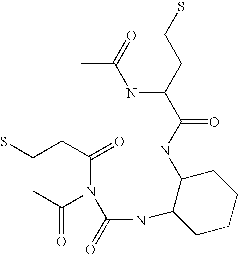
(5) 2-acetylamino-N-[2-(2-acetylamino-4-mercaptobutyrylamino)cyclohexyl]-4-mercaptobutyramide, 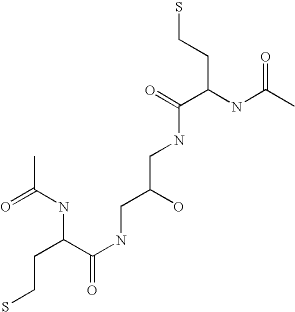
(6) 2-acetylamino-N-[2-(2-acetylamino-4-mercaptobutyrylamino)-2-hydroxypropyl]-4-mercaptobutyramide, and 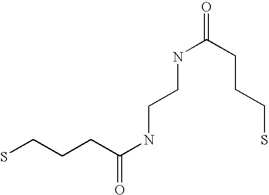
(7) 4-mercapto-N-[2-(4-mercaptobutyrylamino)ethyl]butyramide.

8. The method of claim 7 wherein the keratin material is a keratin fiber.

9. The method according to claim 7 wherein the dithiol is 2-acetylamino-N-{2-[2-(2-acetylamino-4-mercaptobutyrylamino)ethylamino]ethyl}-4-mercaptobutyramide.

10. A method of strengthening a keratin material comprising applying to the keratin material at least one dithiol of formula (III): 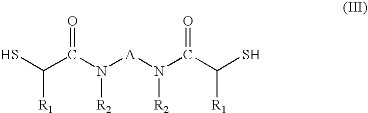
wherein A is chosen from
linear and non-linear, saturated and unsaturated C₁ to C₁₈ alkylene groups, optionally interrupted with at least one entity chosen from aromatic and non-aromatic 5-, 6- and 7-membered rings and from heteroatoms and functional groups, and
5-, 6- and 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups; and
R₁ and R₂, which may be identical or different, are chosen from H and C₁ to C₈ alkyl.

11. The method of claim 10, wherein the keratin material is a keratin fiber.

12. The method of claim 10, wherein the C₁ to C₁₈ alkylene groups of A are interrupted by at least one heteroatom or functional group chosen from: 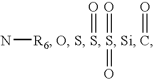
wherein R₆ is chosen from H, (C₁ to C₄)alkyl, (C₁ to C₄)acyl, (C₁ to C₄)alkyloxycarbonyl, and (C₁ to C₄)alkylaminocarbonyl groups

13. A cosmetic composition comprising, in a cosmetically acceptable medium, at least one dithiol of formula (II): 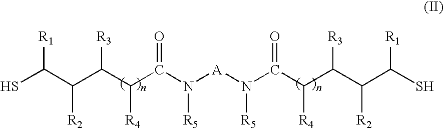
wherein each n, which may be identical or different, is chosen from 0 and 1;
A is chosen from
linear and non-linear, saturated and unsaturated C₁ to C₁₈ alkylene groups, optionally interrupted with at least one entity chosen from 5-, 6- and 7-membered aromatic and non-aromatic rings, heteroatoms and functional groups; and
5-, 6- and 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups;
R₁, R₂, R₃, and R₄, which may be identical or different, are chosen from H, COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups; and
R₅, which may be identical or different, is chosen from H, (C₁ to C₄)alkyl, (C₁ to C₄)acyl, (C₁ to C₄)alkyloxycarbonyl, and (C₁ to C₄)alkylaminocarbonyl groups.

14. The composition of claim 13, wherein the C₁ to C₁₈ alkylene groups of A are interrupted by at least one heteroatom or functional group chosen from: 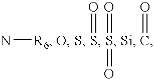
wherein R₆ is chosen from H, (C₁ to C₄)alkyl, (C₁ to C₄)acyl, (C₁ to C₄)alkyloxycarbonyl, and (C₁ to C₄)alkylaminocarbonyl groups.

15. The composition according to claim 13, wherein the at least one dithiol is chosen from 2-acetylamino-N-[2-(2-acetylamino-4-mercaptobutyrylamino)ethyl]-4-mercaptobutyramide (1), 2-acetylamino-N-[2-(2-acetylamino-4-mercaptobutyrylamino)butyl]-4-mercaptobutyramide (2), 2-acetylamino-N-[2-(2-acetylamino-4-mercaptobutyrylamino)hexyl]-4-mercaptobutyramide (3), 2-acetylamino-N-{2-[2-(2-acetylamino-4-mercaptobutyrylamino)ethylamino]ethyl}-4-mercaptobutyramide (4), 2-acetylamino-N-[2-(2-acetylamino-4-mercaptobutyrylamino)cyclohexyl]-4-mercaptobutyramide (5), 2-acetylamino-N-[2-(2-acetylamino-4-mercaptobutyrylamino)-2-hydroxypropyl]-4-mercaptobutyramide (6), and 4-mercapto-N-[2-(4-mercaptobutyrylamino)ethyl]butyramide (7).

16. The composition according to claim 13, wherein the at least one dithiol is 2-acetylamino-N-[2-(2-acetylamino-4-mercaptobutyrylamino)cyclohexyl]-4-mercaptobutyramide (5).

17. The composition according to claim 13, further comprising at least one cosmetic additive or formulation adjuvant chosen from anionic, cationic, amphoteric or nonionic surfactants; nacreous agents; opacifiers; pigments and dyes; oils; waxes; ceramides; organic and mineral UV-screening agents; free-radical scavengers; vitamins; proteins; antidandruff agents; plasticizers; agents for modifying the pH; agents for setting the pH; antioxidants; preserving agents; hair dye precursors; and oxidizing agents.

18. A method of increasing the rigidification of a keratin material comprising applying to the keratin material at least one dithiol compound of formula (I): 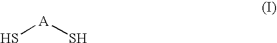
wherein A is chosen from
linear and branched, saturated and unsaturated C₁ to C₃₀ hydrocarbon-based chains which are optionally interrupted with at least one entity chosen from heteroatoms, functional groups, and 5-, 6- and 7-membered aromatic and non-aromatic rings
5-, 6- or 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups.

19. The method of claim 18 wherein the keratin material is a keratin fiber.

20. The method of claim 18, wherein the C₁ to C₃₀ hydrocarbon-based chains of A are interrupted with at least one functional group chosen from: 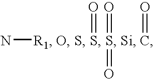
wherein R₁ is chosen from H, (C₁ to C₈)alkyl, (C₁ to C₈)acyl, (C₁ to C₈)alkyloxycarbonyl, (C₁ to C₈)alkylaminocarbonyl, and halo groups.

21. A method of imparting a breaking-resistance effect to hair, comprising applying to the hair a dithiol compound of Formula (I): 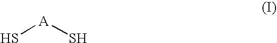
wherein A is chosen from
linear and branched, saturated and unsaturated C₁ to C₃₀ hydrocarbon-based chains which are optionally interrupted with at least one entity chosen from heteroatoms, functional groups, and 5-, 6- and 7-membered aromatic and non-aromatic rings
5-, 6- or 7-membered aromatic and non-aromatic rings,
wherein A is optionally substituted with at least one group chosen from COOH, OH, NH₂, (C₁ to C₈)alkyl, (C₁ to C₈)alkylamino, (C₁ to C₈)acylamino, (C₁ to C₈)acyloxy, (C₁ to C₈)alkyloxycarbonylamino, (C₁ to C₈)alkylaminocarbonyloxy, halo, and (C₁ to C₈)alkylaminocarbonyl groups.

22. The method of claim 21, wherein the C₁ to C₃₀ hydrocarbon-based chains of A are interrupted with at least one functional group chosen from: 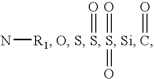
wherein R₁ is chosen from H, (C₁ to C₈)alkyl, (C₁ to C₈)acyl, (C₁ to C₈)alkyloxycarbonyl, (C₁ to C₈)alkylaminocarbonyl, and halo groups.

23. The method of claim 21, wherein the hair is chosen from hair of a person of African descent, embrittled hair, or sensitized hair.
